(19) United States
(12) Patent Application Publication
(10) Pub. No.: US 2004/0043085 A1
(43) Pub. Date: Mar. 4, 2004

(54) Anti-wrinkles emulsion

(76) Inventors: Serena Mautone, Karlsruhe (DE); Martin Blechschmidt, Gondelsheim (DE); Nadja Holland, Karlsruhe (DE); Annette Grill, Stutensee (DE); Jurgen Franke, Karlsruhe (DE); Thomas Brecht, Vellberg (DE)

Correspondence Address: Barry Jacobsen, Legal Division, Warner-Lambert Company LLC, 201 Tabor Road, Morris Plains, NJ, 07950, US

(21) Appl. No.: 10/655,083

(22) Filed: Sep. 4, 2003

(30) Foreign Application Priority Data
Sep. 4, 2002 (DE) 20213787.2

(51) Int. Cl. A61K 31/7048; A61K 31/20; A61K 7/06; A61K 35/78; A61K 31/17

(52) U.S. Cl. 424/729; 514/27; 514/456; 514/559; 424/74; 514/588

(57) ABSTRACT

The present invention relates to a cosmetic composition for the treatment, care and/or protection of the skin. In particular, the present invention relates to a cosmetic composition which can be applied topically and which provides for prevention, reduction and regeneration of skin ageing.

The present invention relates to a cosmetic composition for the treatment, care and/or protection of the skin. In particular, the present invention relates to a cosmetic composition which can be applied topically and which provides for prevention, reduction and regeneration of skin ageing.

The ageing process in the skin results in wrinkles which are caused by various factors. For example, it has been found in investigations that skin ageing is partly genetically determined and is partly based on environmental influences.

Genetically determined skin ageing manifests itself in various symptoms:

reduced production of tallow and perspiration causes a reduction in the number of barrier lipids, which in turn leads to a tendency for skin dryness;

thinning of the skin; and

decrease in the collagen content and degeneration of the elastic fibres leads to sagging of the connective tissue and thus ultimately to reduced elasticity and wrinkling.

Genetic skin ageing is intensified by so-called light-induced skin ageing.

It is known that a prolonged exposure of the skin to UV rays by, for example, sunlight increases the number of wrinkles on the face and on the neck and makes the skin dry and rough.

Investigations have shown that UV radiation on the skin produces free radicals which may lead to mutations and harm cell membranes.

In parallel to this, the distribution of collagen changes, which causes sagging of the connective tissue. This in turn leads to reduced elasticity of the skin and simultaneously to increased wrinkling.

There are already a number of products on the market which aim to counter these effects of skin ageing.

For example, U.S. Pat. No. 6,207,711 describes a skin care composition which comprises a compound which prevents light-induced skin ageing. The compounds which prevent light-induced skin ageing are triterpenoid derivatives, which are derivatives of ursolic acid, oleanolic acid or betulinic acid which are substituted in the 3-position and/or in the 28-position of the ring structure by a functional group, where at least one functional group is a functional group with an aromatic ring.

U.S. Pat. No. 5,723,139 describes a skin conditioning composition which comprises (a) a retinoid compound, which is chosen from retinol, a retinyl ester and mixtures thereof, (b) a polycyclic triterpenecarboxylic acid, which is chosen from oleanolic acid, ursolic acid and glycyrrethinic acid, and (c) a cosmetically acceptable carrier.

The compositions known hitherto do, however, leave room for further improvements of the effects, particularly with regard to a combined effect of regeneration, improvement and prevention of skin ageing, specifically wrinkling, and also an increase in the skin moisture when the cosmetic composition is applied topically.

Consequently, it is an object of the present invention to provide a cosmetic composition for the treatment, care and/or protection of the skin which has an excellent triple action with regard to regeneration, improvement and prevention of skin ageing, specifically wrinkling.

It is also an object of the present invention to provide a cosmetic composition which has the abovementioned effects, where the effective constituents of this cosmetic composition are exclusively vegetable in origin.

The present invention provides a cosmetic composition which comprises (a) an isoflavone, (b) a pentacyclic triterpenoid derivative, (c) an extract of green tea and (d) a cosmetically acceptable carrier.

The isoflavone used as constituent (a) in the cosmetic composition according to the invention is, according to the invention, at least one constituent of the group of plant dyes, most of which are yellowish in colour, derived from the basic structure of isoflavone (=3-phenylchromone, 3-phenyl-4.H-1-benzopyran-4-one) of the following formula (1), also referred to as isoflavonoids:

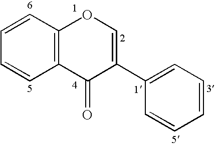

Examples of isoflavones to be used according to the invention are, in addition to isoflavone itself, the following compounds which have the substituents given in the table below on the positions, designated in each case, of the ring backbone of isoflavone of the formula (I) given above:

TABLE 1

| | 5 | 7 | 3′ | 4′ |
|---|---|---|---|---|
| Daidzein | | OH | | OH |
| Genistein | OH | OH | | OH |
| Prunetin | OH | OCH₃ | | OH |
| Biochanin A | OH | OH | | OCH₃ |
| Orobol | OH | OH | OH | OH |
| Santal | OH | OCH₃ | OH | OH |
| Pratensein | H | OH | OH | OH |
| Isoflavone | H | H | H | H |

According to the invention, isoflavones preferably used as constituent (a) include isoflavone, daidzein, genistein, glycitein, prunetin, biochanin A, orobol, santal, pratensein and irigenin, and also mixtures thereof, more preferably glycitein, daidzein and genistein.

These isoflavones can be obtained from natural sources, such as types of clover and/or leguminosae. For example, the isoflavones daidzein and genistein which are used particularly preferably can, for example, be isolated from soya.

The constituent (a) according to the invention is customarily added to the cosmetic composition in an amount of from 0.001 to 10% by weight, usually 0.01 to 5% by weight, preferably 0.05 to 1% by weight, based on the total weight of the cosmetic composition.

The pentacyclic triterpenoid derivative used as constituent (b) in the cosmetic composition according to the invention is, according to the invention, at least one compound from compounds of the following formulae (II), (HI) or (IV), derivatives of these compounds, and salts thereof.

Compounds of the formula (II): (ursolic acid)

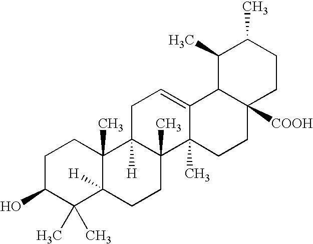

Compounds of the formula (III): (oleanolic acid)

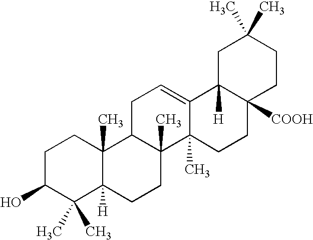

Compounds of the formula (IV): (betulinic acid)

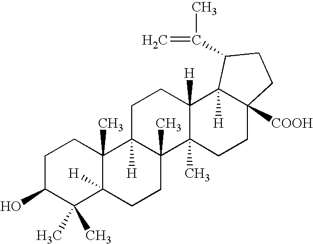

Derivatives of compounds of the above formulae (II), (III) or (IV) include esters, such as the methyl, ethyl, propyl, butyl and benzyl esters, ethers, amides and oximes of ursolic acid, oleanolic acid or betulinic acid, and the salts thereof.

Examples of pentacyclic triterpenoid derivatives of compounds of the formula (II), (III) or (IV) preferably used as constituent (b) in the cosmetic composition according to the invention are, for example, the compounds functionalized on the carboxyl group in position 28 and/or on the hydroxyl group in position 3 of the ursolic acid, oleanolic acid or betulinic acid backbone, mentioned in U.S. Pat. No. 6,207,711, or salts thereof.

Suitable salts of the pentacyclic triterpenoid derivatives used according to the invention are, in particular, the alkali metal and alkaline earth metal salts, ammonium salts and salts of organic amine compounds, in particular the alkali metal salts, such as the sodium and potassium salts.

In a preferred embodiment of the present invention, the alkali metal salts, in particular the sodium or potassium salts, of ursolic and/or oleanolic acid are used.

Pentacyclic triterpenoid derivatives can, for example, be extracted from plants using customarily used organic solvents, such as alcohols, e.g. methanol or ethanol. In addition, they can be prepared by chemical synthesis in a simple manner.

The constituent (b) according to the invention is customarily added in an amount of from 0.0001 to 50% by weight, usually 0.01 to 10% by weight, preferably 0.1 to 5% by weight, based on the total weight of the cosmetic composition.

The constituent (c) in the cosmetic composition according to the invention includes, according to the invention, extracts of green tea. Usually, use is made according to the invention of water-soluble extracts of green tea. Such extracts are known to the person skilled in the art in the relevant specialist area and are available commercially.

The constituent (c) according to the invention is customarily added in an amount of from 0.001 to 30% by weight, usually 0.01 to 15% by weight, preferably 0.5 to 7.5% by weight, based on the total weight of the cosmetic composition.

Without wishing to be tied to any theory, the inventors of the present invention presently assume that the constituents (a) and (c) of the cosmetic composition according to the invention show, as a result of stimulation of the collagen synthesis, a synergistic effect with regard to regeneration, improvement and prevention of skin ageing and wrinkling, which is further improved by the keratinocyte-stimulating property (stimulation of the production of triglycerides and glycoceramides, and of the ceramides and cholesterol esters important for the barrier function) of the pentacyclic triterpenoid derivatives (b) used according to the invention.

Usually, the cosmetic composition according to the invention is used for topical application to the skin. In this connection, topical compositions are understood as meaning those compositions which are suitable for applying active ingredients to the skin in a fine distribution and preferably in a form which can be absorbed by the skin. Suitable for this purpose are, for example, aqueous and aqueous-alcoholic solutions, sprays, foams, foam aerosols, ointments, aqueous gels, emulsions of the O/W or W/O type, microemulsions or cosmetic stick preparations.

The cosmetically acceptable carrier used as constituent (d) in the cosmetic composition according to the invention is water, a liquid based on water, an oil, a gel, an emulsion or microemulsion, a dispersion or a mixture thereof.

According to the invention, it is of importance that the carrier used has good skin compatibility. For topical compositions, aqueous gels, emulsions or microemulsions are particularly advantageous.

In addition, the cosmetic composition according to the invention can comprise further optional constituents.

Emulsifiers which can be used are nonionic surfactants, zwitterionic surfactants, ampholytic surfactants or anionic emulsifiers. The emulsifiers can be present in the composition according to the invention in amounts of from 0.1 to 10, preferably 1 to 5% by weight, based on the total weight of the cosmetic composition.

The nonionic surfactant used may, for example, be a surfactant from at least one of the following groups:

1. addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;

2. C_{12/18} fatty acid mono- and diesters of addition products of from 1 to 30 mol of ethylene oxide onto glycerol;

3. glycerol mono- and diesters and sorbitan mono- and diesters of saturated and unsaturated fatty acids having 6 to 22 carbon atoms and their ethylene oxide addition products;

4. alkyl mono- and oligoglycosides having 8 to 22 carbon atoms in the alkyl radical and ethoxylated analogues thereof;

5. addition products of from 15 to 60 mol of ethylene oxide onto castor oil and/or hydrogenated castor oil;

6. polyol and, in particular, polyglycerol esters, such as, for example, polyglycerol polyricinoleate, polyglycerol poly-12-hydroxystearate or polyglycerol dimerate. Likewise suitable are mixtures of compounds of two or more of these classes of substance;

7. addition products of from 2 to 15 mol of ethylene oxide onto castor oil and/or hydrogenated castor oil;

8. partial esters based on linear, branched, unsaturated or saturated C_{6/22}-fatty acids, ricinoleic acid, and 12-hydroxystearic acid and glycerol, polyglycerol, pentaerythritol, dipentaerythritol, sugar alcohols (e.g. sorbitol), alkylglucosides (e.g. methyl glucoside, butyl glucoside, lauryl glucoside), and polyglucosides (e.g. cellulose);

9. mono-, di- and trialkyl phosphates, and mono-, di- and/or tri-PEG alkyl phosphates and salts thereof;

10. wool wax alcohols;

11. polysiloxane-polyalkyl-polyether copolymers and corresponding derivatives;

12. mixed esters of pentaerythritol, fatty acids, citric acid and fatty alcohol according to DE-A-1 165574 and/or mixed esters of fatty acids having 6 to 22 carbon atoms, methylglucose and polyols, preferably glycerol or polyglycerol, and also

13. polyalkylene glycols

14. betaines

15. ester quats.

In a preferred embodiment of the present invention, the emulsifier system used in the cosmetic composition according to the invention is an ethylene oxide-free emulsifier system based on vegetable raw materials. Emulsifier systems which have proven particularly suitable for this purpose are those based on sucrose stearates and/or polyglycerol stearates with glucosides.

In a particular embodiment, these emulsifier systems are used in the cosmetic composition according to the invention in conjunction with polar and non-polar oils, as described in more detail below. This is because it has been found according to the invention that the incorporation of these constituents aids the effect of the constituents (a) to (c) with regard to their free radical scavenging properties.

In addition, the emulsifiers used may be zwitterionic surfactants. Zwitterionic surfactants is the term used for those interface-active compounds which carry, in the molecule, at least one quaternary ammonium group and at least one carboxylate or sulphonate group. Particularly suitable zwitterionic surfactants are the so-called betaines, such as N-alkyl-N,N-dimethylammonium glycinates, for example cocoalkyldimethylammonium glycinate, N-acylaminopropyl-N,N-limethylammonium glycinates, for example cocoacylamino-propyldimethylammonium glycinate, and 2-alkyl-3-carboxymethyl-3-hydroxyethylimidazolines each having 8 to 18 carbon atoms in the alkyl or acyl group, and cocoacylaminoethyl hydroxyethylcarboxymethylglycinate. Particular preference is given to the fatty acid amide derivative known under the CTFA name Cocamidopropyl Betaine.

Likewise suitable emulsifiers are ampholytic surfactants. Ampholytic surfactants are understood as meaning those surface-active compounds which, apart from containing a C_{8/18}-alkyl or acyl group, contain at least one free amino group and at least one —COOH or —SO₃H group in the molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 carbon atoms in the alkyl group. Particularly preferred ampholytic surfactants are N-cocoalkylaminopropionate, cocoacylaminoethylaminopropionate and C_{12/18}-acylsarcosine. In addition to the ampholytic emulsifiers, quaternary emulsifiers are also suitable, those of the ester quat type, preferably methyl-quaternized difatty acid triethanolamine ester salts, being particularly preferred. In addition, the anionic emulsifiers used may be alkyl ether sulphates, monoglyceride sulphates, fatty acid sulphates, sulphosuccinates and/or ethercarboxylic acids.

Suitable oil bodies are Guerbet alcohols based on fatty alcohols having 6 to 18, preferably 8 to 10 carbon atoms, esters of linear C6-C22 fatty acids with linear C₆-C₂₂ fatty alcohols, esters of branched C₆-C₁₃ carboxylic acids with linear C₆-C₂₂ fatty alcohols, esters of linear C₆-C₂₂-fatty acids with branched alcohols, in particular 2-ethylhexanol, esters of linear and/or branched fatty acids with polyhydric alcohols (such as, for example, propylene glycol, dimerdiol or trimertriol) and/or Guerbet alcohols, triglycerides based on C₆-C₁₀ fatty acids, liquid mono-/di-/triglyceride mixtures based on C₆-C₁₈ fatty acids, esters Of C₆-C₂₂ fatty alcohols and/or Guerbet alcohols with aromatic carboxylic acids, in particular benzoic acid, esters Of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂-fatty alcohol carbonates, Guerbet carbonates, esters of benzoic acid with linear and/or branched C₆-C₂₂ alcohols (e.g. Finsolv© TN), dialkyl ethers, ring-opened products of epoxidized fatty acid esters with polyols, silicone oils and/or aliphatic or naphthenic hydrocarbons. Oil bodies which may be used are also silicone compounds, for example dimethylpolysiloxanes, methylphenylpolysiloxanes, cyclic silicones and amino-, fatty acid-, alcohol-, polyether-, epoxy-, fluorine-, alkyl- and/or glycoside-modified silicone compounds which are either in the form of a liquid or in the form of a resin at room temperature. The oil bodies can be present in the compositions according to the invention in amounts of from 1 to 90% by weight, preferably 5 to 80% by weight, and in particular 10 to 50% by weight, based on the total weight of the cosmetic composition.

According to a preferred embodiment, the cosmetic composition according to the invention further comprises (e) UV light protection filters.

These are soluble compounds or insoluble pigments or mixtures thereof in the case of soluble compounds, UV light protection filters are understood as meaning organic substances which are able to absorb ultraviolet rays and give off the absorbed energy again in the form of longer-wave radiation, e.g. heat. The organic substances may be oil-soluble or water-soluble.

Examples of oil-soluble UV-B filters which can be used are the following substances:

1. 3-benzylidenecamphor and derivatives thereof, e.g. 3-(4-methylbenzylidene)camphor;

2. 4-aminobenzoic acid derivatives, preferably 2-ethylhexyl-4-(dimethylamino)benzoate, 2-octyl-4-(dimethylamino)benzoate and amyl-4-(dimethylamino)benzoate;

3. esters of cinnamic acid, preferably 2-ethylhexyl-4-methoxycinnamate, propyl-4-methoxycinnamate, isoamyl-4-methoxycinnamate, 2-ethylhexyl-2-cyano-3-phenylcinnamate (octocrylene);

4. esters of salicylic acid, preferably 2-ethylhexyl salicylate, 4-isopropylbenzyl salicylate, homomenthyl salicylate;

5. derivatives of benzophenone, preferably 2-hydroxy-4-methoxybenzophenone, 2-hydroxy-4-methoxy-4′-methylbenzophenone, 2,2′-dihydroxy-4-methoxybenzophenone;

6. esters of benzalmalonic acid, preferably di-2-ethylhexyl-4-methoxybenzalmalonate;

7. triazine derivatives, such as, for example, 2,4,6-trianilino-(p-carbo-2′-ethyl-1′-hexyloxy)-1,3,5-triazine and octyltriazone;

8. propane-1,3-diones, such as, for example, 1-(4′-tert-butylphenyl)-3-(4-methoxyphenyl)propane-1,3-dione.

Suitable water-soluble substances are:

1. 2-phenylbenzimidazole-5-sulphonic acid and the alkali metal, alkaline earth metal, ammonium, alkylammonium, alkanolammonium and glucammonium salts thereof;

2. sulphonic acid derivatives of benzophenone, preferably 2-hydroxy-4-methoxy-benzophenone-5-sulphonic acid and its salts;

3. sulphonic acid derivatives of 3-benzylidenecamphor, such as, for example, 4-(2-oxo-3-bornylidenemethyl)benzenesulphonic acid and 2-methyl-5-(2-oxo-3-bornylidene)sulphonic acid and salts thereof.

Particular preference is given to the use of the esters of cinnamic acid, preferably 2-ethylhexyl-4-methoxycinnamate, isopentyl-4-methoxycinnamate, 2-ethylhexyl-2-cyano-3-phenylcinnamate (octocrylene). Furthermore, the use of derivatives of benzophenone, in particular 2-hydroxy-4-methoxybenzophenone, 2-hydroxy-4-methoxy-4′-methylbenzophenone, 2,2′-dihydroxy-4-methoxybenzophenone, and the use of propane-1,3-diones, such as, for example, 1-(4-tert-butylphenyl)-3-(4′-methoxyphenyl)propane-1,3-dione, is preferred.

Suitable typical UV-A filters are, in particular, derivatives of benzoylmethane, such as, for example, 1-(4′-tert-butylphenyl)-3-(4′-methoxyphenyl)propane-1,3-dione, 4-tert-butyl-4′-methoxydibenzoyl methane or 1-phenyl-3-(4′-isopropylphenyl)propane-1,3-dione. The UV-A and UV-B filter materials can of course also be used in mixtures.

Further light protection filters which may also be used, however, are insoluble pigments, e.g. finely dispersed metal oxides or salts, such as, for example, titanium dioxide, zinc oxide, iron oxide, aluminium oxide, cerium oxide, zirconium oxide, silicates (talc), silicon oxide, barium sulphate und zinc stearate, und mixtures thereof The particles here should have an average diameter of less than 100 nm, preferably between 5 und 50 nm and in particular between 15 und 30 nm.

In a particularly preferred embodiment of the present invention, the cosmetic composition according to the invention comprises, in addition to the constituents (a)-(d), as constituent (e) a mixture of titanium dioxide, silicon oxide und iron oxide, for example a mixture of 75-84% by weight of SiO₂, 15-24% by weight of TiO₂ und less than 2% by weight of Fe₂O₃.

Such a product is commercially available, for example, under the name Ronasphere© LDP.

According to the invention, it has been found that the addition of a mixture of titanium dioxide, silicon oxide und iron oxide to the cosmetic composition according to the invention achieves not only a UV filter action, but also a visible reduction in wrinkle depth on the skin following application of the cosmetic composition.

This is attributed to the fact that the incorporation of the mixed oxide of titanium dioxide, silicon dioxide und iron oxide in the cosmetic composition changes the diffraction of light on the skin, giving rise to the visual impression of a reduction in wrinkle depth or a disappearance of the wrinkles.

In addition to the two groups of primary light protection substances mentioned above, it is also possible to use secondary light protection agents of the antioxidant type which interrupt the photochemical reaction chain which is triggered when UV radiation penetrates the skin. Typical examples thereof are superoxide dismutase, tocopherols (vitamin E) und ascorbic acid (vitamin C). The total content of light protection agents in the cosmetic composition according to the invention is usually 1 to 20% by weight, preferably 5 to 15% by weight.

In addition, the cosmetic composition according to the invention can comprise care substances, further cosmetic active ingredients and/or auxiliaries und additives.

Further cosmetic active ingredients which may be added are, in particular, skin humectants, antimicrobial substances and/or deodorizing und antiperspirant substances. This has the advantage that further desired effects can be achieved which contribute to the care or treatment of the skin or, for example, increase the well-being of the user of the cosmetic composition when using this composition.

For example, the cosmetic composition may, inter alia, also comprise physiologically suitable solvents, care constituents, such as, for example, oils, waxes, fats, refatting substances, thickeners und fragrances. A high proportion of care substances is advantageous particularly for the topical prophylactic or cosmetic treatment of the skin. It is particularly advantageous if the composition also comprises further care components in addition to the animal und vegetable fats und oils which in many cases likewise have a care action. The group of care active ingredients which can be used includes, for example, fatty alcohols having 8-22 carbon atoms, in particular fatty alcohols of natural fatty acids; animal und vegetable protein hydrolysates, in particular elastin, collagen, keratin, milk protein, silk protein, oat protein, pea protein, almond protein und wheat protein hydrolysates; vitamins und vitamin precursors; mono-, di- und oligosaccharides; plant extracts; honey extracts; ceramides; phospholipids; vaseline, paraffin und silicone oils; fatty acid und fatty alcohol esters, in particular the monoesters of the fatty acids with alcohols having 3-24 carbon atoms.

Specific examples of softening and care agents which can be incorporated in the cosmetic composition according to the invention are shea butter, karite butter and/or jojoba oil.

Auxiliaries and additives are understood as meaning substances which are suitable for improving the aesthetic, performance and/or cosmetic properties, such as, for example, coemulsifiers, organic solvents, superfatting agents, stabilizers, antioxidants, fats or waxes, bodying agents, thickeners, tanning agents, vitamins, cationic polymers, biogenic active ingredients, preservatives, hydrotopic agents, solubilizers, colourants and fragrances.

In a preferred embodiment of the present invention, the cosmetic composition according to the invention comprises, in addition to the essential constituents (a) to (d), the additional constituent urea for improving the water retention capacity of the skin, in an amount of usually 0.001 to 10% by weight, preferably 0.1 to 7.5% by weight, based on the total weight of the cosmetic composition.

In a preferred embodiment of the present invention, the cosmetic composition according to the invention comprises, in addition to the essential constituents (a) to (d), the additional constituent (f) panthenol for improving the water retention capacity of the skin, in an amount of usually 0.001 to 10% by weight, preferably 0.1 to 7.5% by weight, based on the total weight of the cosmetic composition.

Further ingredients which the cosmetic composition according to the invention may comprise are, in minor amounts, other surfactants compatible with the other ingredients. Typical examples of anionic surfactants are soaps, alkylbenzene sulphonates, alkane sulphonates, olefin sulphonates, alkyl ether sulphonates, glycerol ether sulphonates, α-methyl ester sulphonates, sulphofatty acids, alkyl sulphates, fatty alcohol ether sulphates, glycerol ether sulphates, hydroxy mixed ether sulphates, monoglyceride (ether) sulphates, fatty acid amide (ether) sulphates, mono and dialkyl sulphosuccinates, mono and dialkyl sulphosuccinamates, sulphotriglycerides, amide soaps, ethercarboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulphates, protein fatty acid condensates (in particular wheat-based vegetable products) and alkyl (ether) phosphates. If the anionic surfactants contain polyglycol ether chains, these can preferably have a conventional homologue distribution, but preferably have a narrowed homologue distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. If the nonionic surfactants contain polyglycol ether chains, then these may have a conventional homologue distribution, but preferably have a narrowed homologue distribution. Typical examples of cationic surfactants are quaternary ammonium compounds and ester quats, in particular quaternized fatty acid trialkanolamine ester salts. Typical examples of amphoteric or zwitterionic surfactants are alkylbetaines, alkylamidobetaines, aminopropionates, aminoglycinates, imidazoliniumbetaines and sulphobetaines.

According to one embodiment of the invention, the cosmetic composition is in the form of an emulsion, a dispersion, a suspension, an aqueous surfactant preparation, a milk, a lotion, a cream, a balsam, an ointment, a gel, a granulate, a powder, a stick preparation, a foam, an aerosol or a spray. Such formulations are highly suitable for topical preparations. Suitable emulsions are oil-in-water emulsions and W/O emulsions or microemulsions.

The cosmetic compositions are prepared by processes known in the relevant specialist field using customary devices and complying with the in-process and end-process controls known to the person skilled in the art.

1. Cosmetic composition which comprises (a) an isoflavone, (b) a pentacyclic triterpenoid derivative, (c) an extract of green tea and (d) a cosmetically acceptable carrier.

2. Cosmetic composition according to claim 1, where the isoflavone is a constituent chosen from isoflavone, daidzein, genistein, prunetin, biochanin A, orobol, santal, pratensein and irigenin, and mixtures thereof.

3. Cosmetic composition according to claim 2, where the isoflavone is a constituent chosen from daidzein and genistein, and mixtures thereof.

4. Cosmetic composition according to one of the preceding claims, where the pentacyclic triterpenoid derivative is a constituent chosen from the alkali metal salts, alkaline earth metal salts, ammonium salts and salts of organic amine compounds of ursolic acid, oleanolic acid and/or betulinic acid or mixtures thereof.

5. Cosmetic composition according to one of the preceding claims which also comprises (e) a mixture of titanium dioxide, silicon oxide and iron oxide.

6. Cosmetic composition according to claim 5, where the mixture (e) is a mixture of 75-84% by weight Of SiO₂, 15-24% by weight of TiO₂ and less than 2% by weight of Fe₂O₃.

7. Cosmetic composition according to one of the preceding claims which further comprises urea.
